(19) United States
(12) Patent Application Publication
(10) Pub. No.: US 2024/0091419 A1
(43) Pub. Date: Mar. 21, 2024

(54) Disposable Set

(71) Applicant: Terumo BCT, Inc., Lakewood, CO (US)

(72) Inventors: Aerie THUO, Lakewood, CO (US); Adam HUDDLE, Lakewood, CO (US); Gina Aga, Lakewood, CO (US); Christina Gloor, Lakewood, CO (US); Katherine Colagrosso, Lakewood, CO (US); Bruce Gibbs, Lakewood, CO (US)

(21) Appl. No.: 18/367,555

(22) Filed: Sep. 13, 2023

Related U.S. Application Data
(60) Provisional application No. 63/406,879, filed on Sep. 15, 2022.

(51) Int. Cl. A61M 1/02

(52) U.S. Cl. CPC A61M 1/0218; A61M 2202/0415; A61M 2202/0427; A61M 2202/0429; A61M 2202/0439; A61M 2205/123

(57) ABSTRACT

A disposable bag set for separating discrete volumes of a composite fluid includes a separation bag including the composite fluid to be separated; a first bag in fluid communication with the separation bag, the first bag configured to receive a first component of the composite fluid from the separation bag; and a second bag in fluid communication with the first bag and not the separation bag, the second bag configured to receive a product formed using the first component.

### CROSS-REFERENCE TO RELATED APPLICATIONS

This application claims the benefit of U.S. Provisional Application No. 63/406,879 filed on Sep. 15, 2022. The entire disclosure of the above application is incorporated herein by reference.

### FIELD

The present disclosure generally relates to systems and methods for separating components of a composite fluid.

### BACKGROUND

This section provides background information related to the present disclosure which is not necessarily prior art.

Blood collection and processing play important roles in the worldwide health care system. In conventional large scale blood collection, whole blood is removed from a source or donor or patient or subject, separated into its various blood components via centrifugation, filtration, or elutriation, and the blood components are stored in sterile containers for future infusion into a patient for therapeutic use. The separated blood components typically include fractions comprising red blood cells, white blood cells, platelets, and/or plasma. Separation of whole blood into its components can be performed continuously during collection and/or can be performed subsequent to collection in batches. It is desirable to achieve maximal proportions of the different blood components when separating the whole blood. Further, separation of blood into its various components under highly sterile conditions is critical to many therapeutic applications.

### SUMMARY

This section provides a general summary of the disclosure and is not a comprehensive disclosure of its full scope or all of its features.

In various aspects, the present disclosure provides a disposable bag set for separating discrete volumes of a composite fluid. The disposable bag set may include a separation bag including the composite fluid to be separated; a first bag in fluid communication with the separation bag, the first bag configured to receive a first component of the composite fluid from the separation bag; and a second bag in fluid communication with the first bag and not the separation bag, the second bag configured to receive a product formed using the first component.

In at least one example embodiment, the disposable bag set further includes a third bag in fluid communication with the separation bag and not the first bag or second bag. The third bag may be configured to receive a second component of the composite fluid from the separation bag, where the second component is different from the first component and the product.

In at least one example embodiment, the disposable bag set further includes a fourth bag in fluid communication with the separation bag and not the first bag or the second bag or the third bag. The fourth bag may be configured to receive a third component of the composite fluid from the separation bag, where the third component is different from the first component, the second component, and the product.

In at least one example embodiment, the disposable bag set further includes a collection of tubes connecting the separation bag to the first bag, the second bag, and the fourth bag. The collection of tubes may include a first tube length establishing fluid communication between the separation bag and a connector, a second tube length establishing fluid communication between the connector and the first bag, a third tube length establishing fluid communication between the connector and the third bag, and a fourth tube length establishing fluid communication between the connector and the fourth bag.

In at least one example embodiment, the disposable bag set further includes a fifth bag in fluid communication with the separation bag and not the first bag or the second bag or the third bag. The fifth bag may be configured to receive a fourth component of the composite fluid from the separation bag, where the fourth component is different form the first component, the second component, the third component, and the product.

In at least one example embodiment, a tube length establishes fluid communication between the separation bag and the fifth bag. The tube length may include one of a clamp and a filter, where the clamp is configured to move between an open position and a closed position. In the close position, the clamp may be configured to at least partially occlude movement through the tube length.

In at least one example embodiment, the filter may be a red blood cell leukoreduction filter.

In at least one example embodiment, the composite fluid may include whole blood, the first component may include plasma, and the product formed using the first component may include cryoprecipitate.

In at least one example embodiment, the second component includes platelets.

In at least one example embodiment, the third component includes residual leukocytes.

In at least one example embodiment, the fourth component includes red blood cells.

In at least one example embodiment the disposable bag set further includes a sixth bag in fluid communication with the separation bag. The sixth bag may be configured to aid in the collection of the composite fluid.

In at least one example embodiment, the disposable bag set further includes a collection of tubes connecting the separation bag to the sixth bag. The collection of tubes may include a first tube length establishing fluid communication between the separation bag and a connector and a second tube length establishing fluid communication between the sixth bag and the connector, where the connector is joined to a needle.

In at least one example embodiment, the first tube length includes a clamp. The clamp may be configured to move between an open position and a closed position. In the close position, the clamp may be configured to at least partially occlude movement through the first tube length.

In at least one example embodiment, the second tube length includes a clamp. The clamp may be configured to move between an open position and a closed position. In the close position, the clamp may be configured to at least partially occlude movement through the first tube length.

In at least one example embodiment the collection of tubes further includes a needle injury protector disposed between the connector and the needle.

In at least one example embodiment, a tube length may establish fluid communication between the first bag and the second bag.

In various aspects, the present disclosure provides a disposable bag set for separating whole blood. The disposable bag set may include a separation bag including the whole blood to be separated, a first bag in fluid communication with the separation bag, a second bag in fluid communication with the first bag and not the separation bag, a third bag in fluid communication with the separation bag and the first bag or the second bag, a fourth bag in fluid communication with the separation bag and not the first bag, the second bag, or the third bag, and a fifth bag in fluid communication with the separation bag and not the first bag, the second bag, the third bag, or the fourth bag. The first bag may be configured to receive a plasma from the whole blood held by the separation bag. The second bag may be configured to receive cryoprecipitate formed using the plasma. The third bag may be configured to received platelets from the whole blood held by the separation bag. The fourth bag may be configured to receive residual leukocytes from the whole blood held by the separation bag. The fifth bag may be configured to receive red blood cells from the whole blood held by the separation bag.

In at least one example embodiment, the disposable bag set further includes a sixth bag in fluid communication with the separation bag. The sixth bag may be configured to aid in the collection of the whole blood.

In at least one example embodiment, the disposable bag set further includes a first collection of tubes connecting the separation bag to the first bag, the second bag, and the fourth bag; a fifth tube length establishing fluid communication between the separation bag and the fifth bag; a sixth tube length establishing fluid communication between the first bag and the second bag; and a second collection of tubes connecting the separation bag to the sixth bag. The first collection of tubes may include a first tube length establishing fluid communication between the separation bag and a connector, a second tube length establishing fluid communication between the connector and the first bag, a third tube length establishing fluid communication between the connector and the third bag, and a fourth tube length establishing fluid communication between the connector and the fourth bag. The second collection of tubes may include a first tube length establishing fluid communication between the separation bag and a connector and a second tube length establishing fluid communication between the sixth bag and the connector, the connector being joined to a needle.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a perspective view of an example rotor for an apparatus for separating discrete volumes of a composite fluid in accordance with at least one example embodiment of the present disclosure;

FIG. 2 is a partial cross-sectional view along a diametral plane of an example apparatus for separating discrete volumes of a composite fluid that includes, for example, the rotor illustrated in FIG. 1 in accordance with at least one example embodiment of the present disclosure;

FIG. 3 is a cross-sectional view, along a radial plane, of an example first cavity of the rotor illustrated in FIG. 1 configured to receive and hold a separation bag holding the composite fluid in accordance with at least one example embodiment of the present disclosure;

FIG. 4 is a top-down view of an example disposable bag set that may be used to separate discrete volumes of the composite fluid using, for example, the rotor illustrated in FIG. 1, in accordance with at least one example embodiment of the present disclosure; and

FIG. 5 is a cross-sectional view, along a radial plane, of an example second cavity of the rotor illustrated in FIG. 1 configured to receive and hold a plurality of satellite bags configured to receive one or more blood components as separated from the composite fluid in accordance with at least one example embodiment of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers, and/or sections, these elements, components, regions, layers, and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Various components are referred to herein as “operably associated.” As used herein, “operably associated” refers to components that are linked together in operable fashion and encompasses embodiments in which components are linked directly, as well as embodiments in which additional components are placed between the linked components. “Operably associated” components can be “fluidly associated.” “Fluidly associated” refers to components that are linked together such that fluid can be transported between them. “Fluidly associated” encompasses embodiments in which additional components are disposed between the two fluidly associated components, as well as components that are directly connected. Fluidly associated components can include components that do not contact fluid but contact other components to manipulate the system (e.g., a peristaltic pump that pumps fluids through flexible tubing by compressing the exterior of the tube).

Example embodiments will now be described more fully with reference to the accompanying drawings.

At least one embodiment of the present disclosure relates to an apparatus for concurrently (or simultaneously) separating, for example, by centrifugation, discrete volumes of a composite fluid or liquid (e.g., whole blood). An apparatus for separating whole blood is described, for example, in U.S. application Ser. No. 17/975,180 filed Oct. 27, 2022, published as U.S. Pub. No. 2023/0149668 on May 18, 2023, titled METHODS AND SYSTEM FOR SELECTIVELY COUPLING A BLOOD COLLECTION PRESSURE, and listing Jeremy Parsons and Jesse Janzen as inventors and/or U.S. Pat. No. 11,013,850 issued May 25, 2021, titled COMPOSITE FLUID SEPARATION, and listened Bruce W. Gibbs, Bruce Ellingboe, and Jeffrey J. Blakeslee as inventors, the entire disclosures of which are hereby incorporated by reference.

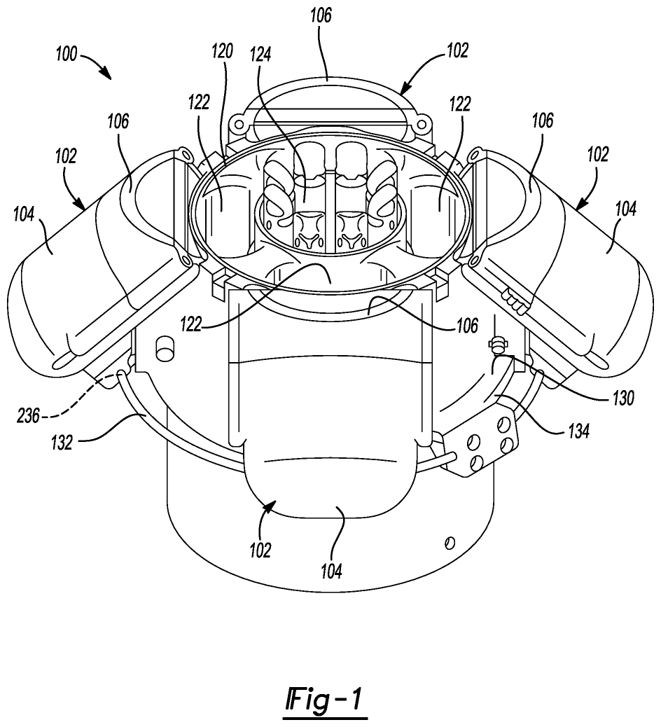

An example rotor 100 for use in an apparatus 200 for separating discrete volumes of a composite fluid is illustrated in FIG. 1. As illustrated the rotor 100 includes one or more separation cells 102, each of the one or more separation cells 102 is configured to receive and hold a separation bag (which may also be referred to a whole blood bag) 400 that includes a volume of composite fluid to be separated. As illustrated, in at least one example embodiment, the rotor 100 may include four separation cells 102, and the rotor 100 may be configured to receive four separation bags 400.

The separation cells 102 may each include a container (which may also be referred to as a bucket) 104 that defines a cavity 230 configured to receive and hold the respective separation bag 400. In at least one example embodiment, each of the separation cells 102 includes a hinged lid (which may also be referred to as a top or upper wall) 106 that allows access to the container 104 for placement or removal of the separation bags 400. The cavity (which may also be referred to as a separation compartment) 230 may also include a lower wall 105 opposite of the upper wall 104 and disposed nearer to a turntable 130. In at least one example embodiment, the upper and lower walls 104, 105 may converge towards a central median axis 226 to define the container 104. In other example embodiments, the cavity 230 may include lateral walls connecting the upper wall 104 and the lower wall 105. Generally, the cavity 230 may be shaped and dimensioned to loosely accommodate the separation bag 400.

The rotor 100 further includes a central container 120 that includes a plurality of satellite containers 122 arranged about a central cavity 124. The satellite containers 122 are each configured to receive and hold one or more satellite bags 502. As illustrated, in at least one example embodiment, the rotor 100 may include four satellite containers 122 disposed around the central cavity 124 and corresponding with each of the four illustrated separation cells 102. As discussed further below, the satellite bags 502 are each configured to receive one or more separated components from the respective separation bag 400. The respective separation bags and satellite bags may be together referred to as a bag system.

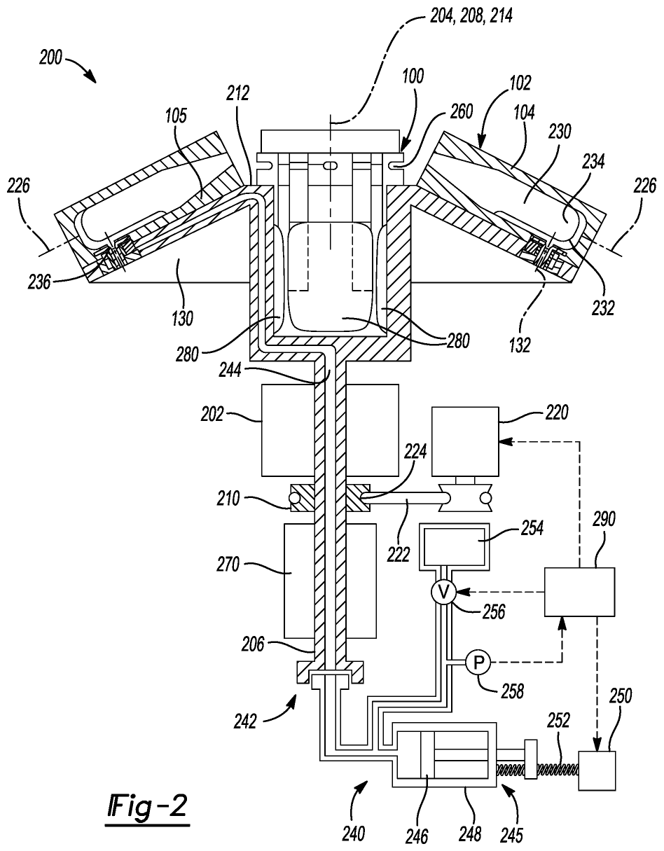

FIG. 2 is a partial cross-sectional view along a diametral plane of the apparatus 200 for separating discrete volumes of a composite fluid including the example rotor illustrated in FIG. 1. As illustrated, the rotor 100 may be supported by a bearing assembly 202 to facilitate rotation of the rotor 100 around a first axis (which may also be referred to as a rotation axis) 204. The rotor 100 may be connected with the bearing assembly 202 via a rotor shaft 206 that extends along a second axis (which may also be referred to as a shaft axis or a shaft longitudinal axis) 208. For example, as illustrated, the central container 120 may be connected to an upper end 212 of the rotor shaft 206, such that the second axis 208 coincides with the first axis 204 and also a third axis (which may also be referred to as a container axis or a container longitudinal axis) 214 of the central container 120. The apparatus 200 may include a motor 220 that facilitates rotation of the rotor 100 about the rotation axis 204. In at least one example embodiment, as illustrated, the motor 220 may interact with the rotor 202 via a belt 222 and pulley 210 system, where the belt 222 is engaged in a groove 224 of the pulley 210 and the pulley 210 is connected to the rotor shaft 206.

Each of the separation cells 102 may define a median longitudinal axis 226. The separation cells 102 may be mounted on a turntable 130 such that the respective median longitudinal axes 226 intersect the rotation axis 204, each of the separation cells 102 are located at the same distance from the rotation axis 204, and/or angles between the different median longitudinal axes 226 are substantially the same (e.g., about 90 degrees). The positioning of the separation cells 102 on the turntable 130 may be adjusted so that the weight on the turntable 130 is equally distributed when the separation cells 102 are empty, for example, to balance the rotor 100.

The apparatus 200 includes a component transferring means for transferring at least one separated component from each separation bag 400 into one or more satellite bags 502 connected to the respective separation bag 400. In at least one example embodiment, the component transferring means includes a squeezing system for squeezing the separation bag 400 as received by and held in the respective containers 104 and causing the transfer of the separated components into the separation bag 400. The squeezing system may include one or more flexible diaphragms (which may also be referred to as “bladders”) 232 selectively coupled to one or more of the containers 104, defining expandable chambers 234 within the cavity 230. For example, as illustrated, the flexible diaphragms 232 may be dimensioned to line the bottom walls of the respective cavities 230, where the bottom walls are those closest to the turntable 130.

The squeezing system may include a manifold 132 that is arranged near a periphery 134 of the turntable 130. For example, each of the expandable chambers 234 may be fluidly connected to the manifold 132 by a supply channel 236. The manifold 132 may have a generally circular shape or arrangement.

The squeezing system may include a hydraulic pumping station 240 that is configured to pump a hydraulic liquid in and/or out of each of the expandable chambers 234. The hydraulic liquid may be selected to have a density that is greater than a density of the densest of the components in the composite liquid to be separated (which would be the red blood cells, when the composite liquid is whole blood). As a result, during centrifugation, the hydraulic liquid within each of the expandable chambers 234, whatever the volume thereof, will generally remain in the most external part of each of the cavities 230.

As illustrated, in at least one example embodiment, the hydraulic pumping station 240 may be connected to the expandable chambers 234 by a rotary seal or fluid coupling 242; a duct 244 that extends through the rotor shaft 206 and also the bottom wall 105 (and/or optionally, the lateral wall(s)) of the central container 120; and, from the central container 120, radially through the turntable 130 where it connects with the manifold 132. In at least one example embodiment, the hydraulic pumping station 240 may include a piston pump 245. The piston pump 245 may include a piston 246 that is movable in a hydraulic cylinder 248. The hydraulic cylinder 248 may be fluidly connected to the expandable chambers 234 via the rotary seal 242 and the duct 244. The piston 246 may be actuated by a stepper motor 250 configured to move a lead screw 252 that is linked to a rod of the piston 246.

The squeezing system may also include a hydraulic liquid reservoir 254. The hydraulic cylinder 248 may be connected to a hydraulic liquid reservoir 254 and access to the hydraulic liquid reservoir 254 by the hydraulic cylinder 248 may be controlled by a valve 256. The valve 256 may be configured to selectively allow the introduction or the withdrawal of hydraulic liquid into and out of a hydraulic circuit define by the hydraulic cylinder 248, the duct 244, and the expandable chambers 234. In at least one example embodiment, a pressure gauge 258 may be connected to the hydraulic circuit and may be configured to measure hydraulic pressure therein.

In at least one example embodiment, the apparatus 200 includes a first balancing means configured to (initially) balance the rotor 100 when the weights of the separation bags 400 placed in the separation cells 102 are different. The first balancing means may include substantially the same structural elements as the elements of the component transferring means. For example, the first balancing means may include four expandable hydraulic chambers similar to hydraulic chamber 234 interconnected by a manifold similar to the manifold 132 and a hydraulic pumping station similar to the hydraulic pumping station 240 for pumping hydraulic liquid into the hydraulic chambers through a duct similar to the duct 244 connected to the manifold. In order to initially balance the rotor 100, whose four separation cells 102 may contain four discrete volumes of a composite fluid having different weights, the hydraulic pumping station may be controlled so as to pump into the interconnected hydraulic chambers, at the onset of a separation process, a predetermined volume of hydraulic liquid that is so selected as to balance the rotor 100. For example, for whole blood, the determination of this balancing volume may take into account the maximum difference in volume between two blood donations and also the maximum difference in hematocrit between two blood donations. Under centrifugation forces, the hydraulic liquid will distribute unevenly in the four separation cells 102 depending on the difference in weight of the separation bags and balance the rotor 100. To achieve a desired or optimal initial balancing, the volume of the cavity 230 of the separation cells 102 may be selected so that the cavities 230, whatever the volume of the separation bags contained therein, are not full after the determined amount of hydraulic liquid has been pumped into the interconnected expandable chambers 234.

In at least one example embodiment, the apparatus 200 may include a second balancing means configured to balance the rotor 100 when the weights of the components transferred into satellite bags 502 in the central container 120 are different. For example, when two blood donations have the same hematocrit and different volumes, the volumes of plasma extracted from each donation may be different, and the same is true when two blood donations have the same volume and different hematocrit. In at least one example embodiment, the second balancing means may include, as illustrated, flexible rectangular pouches 280 that are interconnected by tube sections. For example, the tube sections may connect two adjacent pouches 280 by the bottom thereof. The pouches 280 may contain a volume of balancing liquid having a density close to the density of the composite liquid. The pouches 280 may be dimensioned as to line the inner surface of the central container 120 and to have an internal volume that is larger than the volume of balancing liquid so that the balancing liquid can freely expand in any of the pouches 280. In operation, if, for example, four satellite bags 502 respectively adjacent to the four pouches 280 receive different volumes of a plasma component, the four satellite bags 502 will press unevenly, under centrifugation forces, against the four pouches 280 which will result in the balancing liquid becoming unevenly distributed in the four pouches 280, compensating for the difference in weight in the satellite bags.

In at least one example embodiment, the apparatus 200 may include a controller 290 including a control unit and a memory. The control unit may include, for example, a microprocessor, a controller, and/or the like. The memory may include, for example, a computer readable memory. The memory may provide the control unit with information and programmed instructions relative to various separation protocols (e.g., a protocol for the separation of a plasma component and a blood cell component and/or a protocol for the separation of a plasma component, a platelet component, and a red blood cell component) and to the operation of the apparatus in accordance with such separation protocols. The control unit may be programed to receive, directly or through the memory, information regarding the centrifugation speed(s) at which the rotor 100 is to be rotated during the various stages of a separation process (e.g., stage of component separation, stage of a plasma component expression, stage of suspension of platelets in a plasma fraction, stage of a platelet component expression, etc.), and/or information regarding various transfer flow rates at which separated components are to be transferred from the separation bag 400 into the satellite bags 502. The information relative to the various transfer flow rates can be expressed, for example, as hydraulic liquid flow rates in the hydraulic circuit, and/or as rotation speeds of the stepper motor 250 of the hydraulic pumping station 240. The control unit may be programed to receive, directly or through the memory, information from the pressure gauge 258 and/or for controlling the motor 220 and/or the stepper motor 250 of the hydraulic pumping station 240 to cause the separation apparatus to operate along a selected separation protocol.

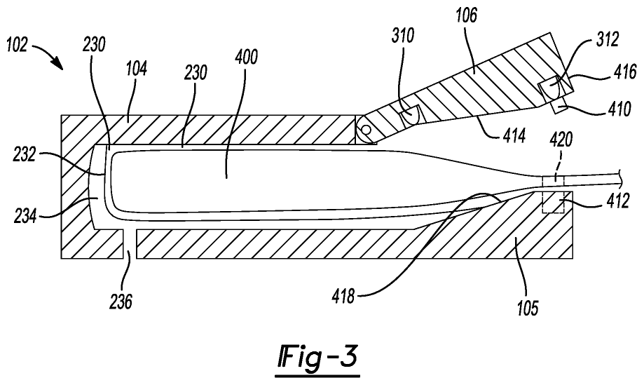

FIG. 3 is a cross-sectional view, along a radial plane, of one of the separation cells 102. As illustrated, one or more sensors 310, 312 may be incorporated into the upper wall 106 and/or the lower wall 105 of the container 104. For example, in at least one example embodiment, the separation cell 102 may include a bag sensor (which may also be referred to as a first sensor) 310 and a tube sensor (which may also be referred to as a second sensor) 312. The sensors 310, 312 may be formed or mounted along the median longitudinal axis 226 of the container 104. The bag sensor 210 may be located further from the rotation axis 204 than the tube sensor 312. In at least one example embodiment, when a separation bag 400 rests in the container 104 and the lid 106 is closed, the bag sensor 310 may face an upper triangular part of the separation bag 400 and the tube sensor 312 may face a proximal end of one or more tube 702, 716 joining together the separation cell 102 and the plurality of satellite bags 502. The bag sensor 310 may be configured to detect blood cells in fluid. The tube sensor 312 may be configured to detect the presence of absence of liquid in the tube as well as to detect blood cells in a liquid. Each of the sensors 310, 312 may include, for example, a photocell including an infrared LED and a photo-detector. In at least one example embodiment, electric power may be supplied to the sensors 310, 312 through a slip ring array 270 that is mounted around the lower portion of the rotor shaft 206, as illustrated in FIG. 2.

As further illustrated in FIG. 3, the containers 104 may include one or more pins 410 and one or more respective recesses 412 for securing the separation bag 400 within the separation cell 102. The pins 410 may protrude from an internal surface 414 of the lid 106 near an opening edge or top 416 of the separation cell 102. The recesses 412 may be defined in an internal surface 418 of the lower surface 105 of the container 104. In at least one example embodiment, the pins 410 may be spaced apart and dimensioned to be received in one or more respective holes 420 in an upper edge (e.g., the two upper corners) of the separation bag 400.

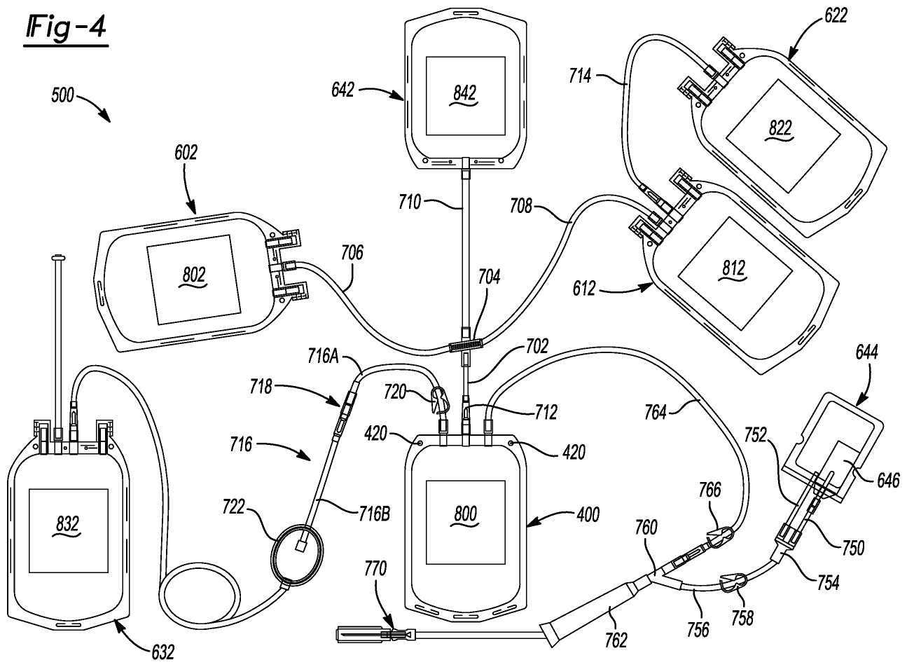

The apparatus 200 is configured to be used with a set of bags (which may also be referred to as a collection of bags or bag set or disposable bag set or disposable set) 500. The separation bag 400 may be used successively for collection separately or away from the apparatus 200 and also for separation within the apparatus 200, while the satellite bags 502 may be used within the apparatus 200 for receipt of separated components. FIG. 4 is a top-down view of an example disposable bag set 500 that may be used to separate discrete volumes of a composite fluid using, for example, the rotor 100 illustrated in FIG. 1. The disposable bag set 500 includes the separation bag 400 configured to receive a discrete volume of whole blood from a source or donor or patient or subject and the one or more satellite bags 502 configured to receive separated components from the separation bag 400.

In at least one example embodiment, the satellite bags 502 may include a first bag 602 configured to receive and store a first component from the composite fluid of the separation bag 400 and a second bag 612 configured to receive and store a second component (distinct from the first component) from the composite fluid of the separation bag 400. The first component may include platelets and the first bag 602 may be a platelet collection bag (interim platelet unit (IPU) bag). The second component may include plasma and the second bag 612 may be a plasma collection bag. The satellite bags 502 may also include a third bag 622 configured to receive and store a third component subsequently separated from the second component. For example, the third component may be cryoprecipitate prepared from plasma and the third bag 622 may be a plasma cryoprecipitate bag or a plasma cryoprecipitate reduced bag.

In at least one example embodiment, the satellite bags 502 may include a fourth bag 632 in addition to the first bag 602, the second bag 612, and the third bag 622. The fourth bag 632 may be configured to receive and store a fourth component (distinct form the first and second components) from the composite fluid of the separation bag 400. For example, the fourth component may include red blood cells (RBC) and the fourth bag 632 may be a red blood cell collection bag (which may also be referred to as a red blood cell storage bag).

In at least one example embodiment, the satellite bags 502 may include a fifth bag 642 in addition to the first bag 602, the second bag 612, and the third bag 622 and optionally the fourth bag 632. The fifth bag 642 may be configured to receive and store a fifth component (distinct from the first, second, and fourth components) from the composite fluid of the separation bag 400. For example, the fifth component may include leukocytes and the fifth bag 642 may be a residual leukocyte bag 642.

The separation bag 400 may be connected to the one or more satellite bags 502 by a tubing system including one or more tubes 702, 706, 708, 710, 714, 716 and one or more connectors 704, 712, 718. For example, in at least one example embodiment, a first end of a first tube length 702 may be joined to (or formed integrally with) the separation bag 400. The first end of the first tube length 702 may be joined the separation bag 400 by a first connector (which may also be referred to as a first coupling) 712. The first connector 712 may include a frangible connector. A second end of the first tube length 702 away from the separation bag 400 may be joined to (or formed integrally with) a second connector (which may also be referred to as a second coupling) 704. The second connector 704 may join the first tube length 702 to a first end of a second tube length 706, where a second end of the second tube length 706 away from the first end may be joined (or formed integrally with) to the first bag 602. The first connector 704 may also join the first tube length 702 to a first end of a third tube length 708, where a second end of the third tube length 708 away from the first end may be joined (or formed integrally with) to the second bag 612. The first connector 704 may also join the first tube length 702 to a first end of a fourth tube length 710, where a second end of the fourth tube length 710 away from the first end may be joined to (or formed integrally with) the fifth bag 642. The first connector 704 may include a cross connector.

A first end of a fourth tube length 714 may be joined to (or formed integrally with) second bag 612, where a second end of the fourth tube length 714 away from the first end may be joined to (or formed integrally with) the third bag 622. A first end of a fifth tube length 716 may be joined to (or formed integrally with) the separation bag 400, where a second end of the fifth tube length 716 may be joined to (or formed integrally with) the fourth bag 632. The fifth tube length 716 may include a filter 722 and/or a first clamp 720. The filter 722 may be a red blood cell leukoreduction filter configured to remove leukocytes from the red cell product. For example, in at least one example embodiment, the filter 722 may be a red blood cell leukoreduction filter configured to remove a sufficient number of leukocytes from the red cell product to meet, for example, as defined by various regulatory bodies. The first clamp 720 may be configured to move between a first or open position and/or a second or intermediate position and/or a third or closed position. In the first position, the first clamp 720 may apply no or minimal pressure to the fifth tube length 716 and fluid may flow freely therethrough. In the intermediate position, the first clamp 720 may apply a closing pressure to the fifth tube length 716 to at least partially obscure fluid flow therethrough. In the closed position, the first clamp 720 may apply a closing pressure to the fifth tube length 716 to completely obscure fluid flow therethrough. The first clamp 720 may be positioned at any point along the fifth tube length 716. For example, in at least one example embodiment, the first clamp 720 may be disposed closer to the second end of the fifth tube length 716 (and the fourth bag 632) than the first end of the fifth tube length 716. In other embodiments, the first clamp 720 may be disposed closer to the first end of the fifth tube length 716 (and the separation bag 400) than the second end of the fifth tube length 716. In still other embodiments, the first clamp 720 may be disposed about halfway along the fifth tube length 716 between the first and second ends. Although not illustrated, it should be appreciated that, in various embodiments, the first tube length 702, the second tube length 706, the third tube length 708, and/or the fourth tube length 710 may similarly include one or more clamps and/or one or more filters.

In at least one example embodiment, the fifth tube length 716 may include a first section 716A and a second section 716B joined by a third connector (which may also be referred to as a third coupling) 718. The configuration may allow fluid to flow through the tubing and the filter only when the connector 718 is in an open state, for example, as set by the operator or user or administrator post-processing. In at least one example embodiment, the connector 718 may include a frangible component or member. Although illustrated as including two or more tube length sections 716A, 716B, it should be appreciated that, in at least one example, embodiment the fifth tube length 716 may be a single tube length, omitting the third connector 718. Similarly, it should be appreciated that although illustrated as single tube lengths, in various embodiments, the first tube length 702, the second tube length 706, the third tube length 708, and/or the fourth tube length 710 may include one or more tube length sections joined by one or more connectors.

In at least one example embodiment, the disposable bag set 500 may further include a sixth bag 644. The sixth bag 644 may be a sample/diversion bag configured to divert a small volume of blood from the source or donor or patient or subject. The sixth bag 644 may include a sample tube holder/luer adapter 646. The sample tube holder/luer adapter 646 may be configured to remove a sample or samples from the diverted volume in the sixth bag 644. The sample tube holder/luer adapter 646 may be joined to (or formed integrally with) a first end of a sixth tube length 750 and an interior volume of the sixth bag 644 may be joined to (or formed integrally with) a first end of a seventh tube length 752, where a second end of the sixth tube length 750 away from the first end and a second end of the seventh tube length 752 away from the first end are joined together by a fourth connector (which may also be referred to a fourth coupling) 754.

The fourth connector 754 may couple the sixth tube length 750 and the seventh tube length 752 to a first end of an eighth tube length 756. The fourth connector 754 may be a Y-connector. The eighth tube length 756 may include a second clamp 758. The second clamp 758 may be configured to move between a first or open position and/or a second or intermediate position and/or a third or closed position. In the first position, the second clamp 758 may apply no or minimal pressure to the eighth tube length 756 and fluid may flow freely therethrough. In the intermediate position, the second clamp 758 may apply a closing pressure to the eighth tube length 756 to at least partially obscure fluid flow therethrough. In the closed position, the second clamp 758 may apply a closing pressure to the eighth tube length 756 to completely obscure fluid flow therethrough. The second clamp 758 may be positioned at any point along the eighth tube length 756. For example, in at least one example embodiment, the second clamp 758 may be disposed closer to the second end of the eighth tube length 756 (and the fifth connector 760) than the first end of the eighth tube length 756. In other embodiments, the second clamp 758 may be disposed closer to the first end of the eighth tube length 756 (and the sixth bag 644) than the second end of the eighth tube length 756. In still other embodiments, the second clamp 758 may be disposed about halfway along the eighth tube length 756 between the first and second ends.

A second end of the eighth tube length 756 may be joined to a fifth connector 760. The fifth connector 760 may also be joined to a needle injury protector (NIP) 762 and a ninth tube length 764. The needle injury protector 762 may be joined to (or formed integrally with) a needle 770 that may be used to draw whole blood from the source or donor or patient or subject. A first end of the ninth tube length 764 may be joined to the separation bag 400, while a second end of the ninth tube length 764 away from the first end is joined to the fifth connector 760. The ninth tube length 764 ay include a third clamp 766. The third clamp 766 may be configured to move between a first or open position and/or a second or intermediate position and/or a third or closed position. In the first position, the third clamp 766 may apply no or minimal pressure to the ninth tube length 764 and fluid may flow freely therethrough. In the intermediate position, the third clamp 766 may apply a closing pressure to the ninth tube length 764 to at least partially obscure fluid flow therethrough. In the closed position, the third clamp 766 may apply a closing pressure to the ninth tube length 764 to completely obscure fluid flow therethrough. The third clamp 766 may be positioned at any point along the ninth tube length 764. For example, in at least one example embodiment, the third clamp 766 may be disposed closer to the second end of the ninth tube length 764 (and the fifth connector 760) than the first end of the ninth tube length 764. In other embodiments, the third clamp 766 may be disposed closer to the first end of the ninth tube length 764 (and the separation bag 400) than the second end of the ninth tube length 764. In still other embodiments, the third clamp 766 may be disposed about halfway along the ninth tube length 764 between the first and second ends.

As recognized by the skilled artisan, the separation bag 400 and also the satellite bags 502 (including the first bag 602, the second bag 612, the third bag 622, the fourth bag 632, and/or the fifth bag 642) may include one or more holes or openings for supporting, positioning, and/or hanging the bags, including for example, the one or more holes 420 in the separation bag 400 for receiving the pins 410 of the containers 104. The separation bag 400 and also the satellite bags 502 (including the first bag 602, the second bag 612, the third bag 622, the fourth bag 632, and/or the fifth bag 642) may be configured to receive one or more identifying labels. For example, as illustrated, the separation bag 400 may include a first label 800, the first bag 602 may include a second label 802, the second bag 612 may include a third label 812, the third bag 622 may include a fourth label 822, the fourth bag 632 may include a fifth label 832, and the fifth bag 642 may include a sixth label 842.

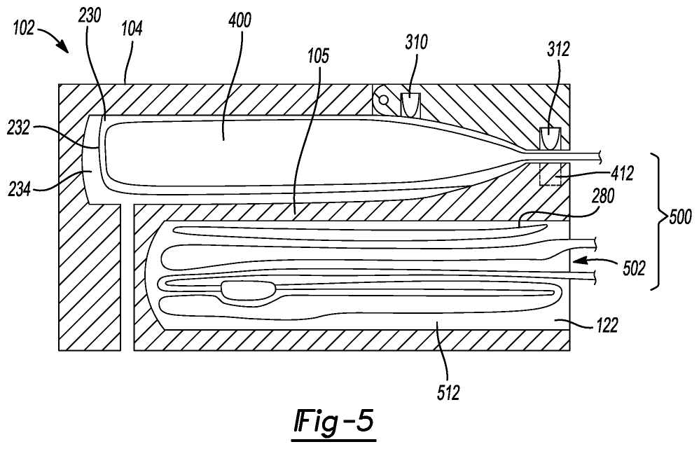

FIG. 5 is a cross-sectional view, along a radial plane, of one of the satellite containers 122. As illustrated, the satellite container 122 includes a cavity 512 configured to receive the plurality of satellite bags 502 including the first bag 602, the second bag 612, the third bag 632, the fourth bag 642, and the fifth bag 644.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A disposable bag set for separating discrete volumes of a composite fluid, the disposable bag set comprising:
a separation bag including the composite fluid to be separated;
a first bag in fluid communication with the separation bag, the first bag configured to receive a first component of the composite fluid from the separation bag; and
a second bag in fluid communication with the first bag and not the separation bag, the second bag configured to receive a product formed using the first component.

2. The disposable bag set of claim 1, wherein the disposable bag set further includes:
a third bag in fluid communication with the separation bag and not the first bag or second bag, the third bag configured to receive a second component of the composite fluid from the separation bag, the second component being different from the first component and the product.

3. The disposable bag set of claim 2, wherein the disposable bag set further includes:
a fourth bag in fluid communication with the separation bag and not the first bag or the second bag or the third bag, the fourth bag configured to receive a third component of the composite fluid from the separation bag, the third component being different from the first component, the second component, and the product.

4. The disposable bag set of claim 3, wherein the disposable bag set further includes:
a collection of tubes connecting the separation bag to the first bag, the second bag, and the fourth bag, the collection of tubes including:
a first tube length establishing fluid communication between the separation bag and a connector;
a second tube length establishing fluid communication between the connector and the first bag;
a third tube length establishing fluid communication between the connector and the third bag; and
a fourth tube length establishing fluid communication between the connector and the fourth bag.

5. The disposable bag set of claim 3, wherein the disposable bag set further includes:
a fifth bag in fluid communication with the separation bag and not the first bag or the second bag or the third bag, the fifth bag configured to receive a fourth component of the composite fluid from the separation bag, the fourth component being different form the first component, the second component, the third component, and the product.

6. The disposable bag set of claim 5, wherein a tube length establishes fluid communication between the separation bag and the fifth bag, the tube length including one of a clamp and a filter, the clamp configured to move between an open position and a closed position, and in the close position, the clamp is configured to at least partially occlude movement through the tube length.

7. The disposable bag set of claim 6, wherein the filter is a red blood cell leukoreduction filter.

8. The disposable bag set of claim 5, wherein the composite fluid includes whole blood, the first component includes plasma, and the product formed using the first component includes cryoprecipitate.

9. The disposable bag set of claim 8, wherein the second component includes platelets.

10. The disposable bag set of claim 9, wherein the third component includes residual leukocytes.

11. The disposable bag of claim 10, wherein the fourth component includes red blood cells.

12. The disposable bag set of claim 3, wherein the disposable bag set further includes a sixth bag in fluid communication with the separation bag and configured to aid in the collection of the composite fluid.

13. The disposable bag set of claim 12, wherein the disposable bag set further includes:
a collection of tubes connecting the separation bag to the sixth bag, the collection of tubes including:
a first tube length establishing fluid communication between the separation bag and a connector; and
a second tube length establishing fluid communication between the sixth bag and the connector, the connector being joined to a needle.

14. The disposable bag set of claim 13, wherein the first tube length includes a clamp, the clamp configured to move between an open position and a closed position, and in the close position, the clamp is configured to at least partially occlude movement through the first tube length.

15. The disposable bag set of claim 13, wherein the second tube length includes a clamp, the clamp configured to move between an open position and a closed position, and in the close position, the clamp is configured to at least partially occlude movement through the first tube length.

16. The disposable bag set of claim 13, wherein the collection of tubes further includes:
a needle injury protector disposed between the connector and the needle.

17. The disposable bag set of claim 1, wherein a tube length establishes fluid communication between the first bag and the second bag.

18. A disposable bag set for separating whole blood, the disposable bag set comprising:
a separation bag including the whole blood to be separated;
a first bag in fluid communication with the separation bag, the first bag configured to receive a plasma from the whole blood held by the separation bag;
a second bag in fluid communication with the first bag and not the separation bag, the second bag configured to receive cryoprecipitate formed using the plasma;
a third bag in fluid communication with the separation bag and the first bag or the second bag, the third bag configured to received platelets from the whole blood held by the separation bag;
a fourth bag in fluid communication with the separation bag and not the first bag, the second bag, or the third bag, the fourth bag configured to receive residual leukocytes from the whole blood held by the separation bag; and
a fifth bag in fluid communication with the separation bag and not the first bag, the second bag, the third bag, or the fourth bag, the fifth bag configured to receive red blood cells from the whole blood held by the separation bag.

19. The disposable bag set of claim 18, wherein the disposable bag set further includes:
a sixth bag in fluid communication with the separation bag and configured to aid in the collection of the whole blood.

20. The disposable bag set of claim 19, wherein the disposable bag set further includes:
a first collection of tubes connecting the separation bag to the first bag, the second bag, and the fourth bag, the first collection of tubes including:
a first tube length establishing fluid communication between the separation bag and a connector;
a second tube length establishing fluid communication between the connector and the first bag;
a third tube length establishing fluid communication between the connector and the third bag; and
a fourth tube length establishing fluid communication between the connector and the fourth bag;
a fifth tube length establishing fluid communication between the separation bag and the fifth bag;
a sixth tube length establishing fluid communication between the first bag and the second bag; and
a second collection of tubes connecting the separation bag to the sixth bag, the second collection of tubes including:
a first tube length establishing fluid communication between the separation bag and a connector; and
a second tube length establishing fluid communication between the sixth bag and the connector, the connector being joined to a needle.
